(19) United States
(12) Patent Application Publication
(10) Pub. No.: US 2016/0120909 A1
(43) Pub. Date: May 5, 2016

(54) Compositions And Methods For The Treatment Of Tinnitus

(71) Applicants: CELL-INNOVATIONS IP PTY LTD, Liverpool, New South Wales (AU); RUSTY PROPERTY HOLDINGS PTY LTD, Liverpool, New South Wales (AU)

(72) Inventors: Wayne Thomas, Liverpool, NSW (AU); Ralph Bright, Liverpool, NSW (AU)

(73) Assignee: Cell-Innovations IP Pty Ltd, Liverpool, NSW (AU)

(21) Appl. No.: 14/890,353

(22) PCT Filed: May 9, 2014
(86) PCT No.: PCT/AU2014/000504

§ 371 Date: Nov. 10, 2015

(30) Foreign Application Priority Data
May 10, 2013 (AU) 2013901658

(51) Int. Cl. A61K 35/28; A61K 35/16; A61K 9/00; A61K 35/44

(52) U.S. Cl. CPC A61K 35/28; A61K 35/44; A61K 35/16; A61K 9/0019; A61K 2035/124

(57) ABSTRACT

In one embodiment, the present invention relates to a method for treating tinnitus in a subject including administering to the subject a composition including a stromal vascular fraction from adipose tissue. In another embodiment, the present invention relates to a method for treating tinnitus in a subject including administering to the subject a composition including a bone marrow cellular fraction. In another embodiment, the present invention relates to a method of treating tinnitus in a subject including administering to the subject a composition including adult stem cells.

### CROSS REFERENCE TO RELATED APPLICATIONS

This application claims priority from Australian Provisional Application No 2013901658 filed 10 May 2013, the contents of which are incorporated herein by reference in their entirety.

### FIELD OF THE INVENTION

The present invention relates to compositions and methods for treatment of tinnitus. In particular, the present invention relates to the treatment of tinnitus by administering compositions comprising stem cells.

### BACKGROUND OF THE INVENTION

Any discussion of the prior art throughout the specification should in no way be considered as an admission that such prior art is widely known or forms part of the common general knowledge in the field.

Tinnitus is the perception of sound within the human ear in the absence of corresponding external sound. Tinnitus is usually not itself a disease, but rather a secondary manifestation of disease or injury to the auditory system. Tinnitus occurs in varying degrees of severity, ranging from a minor, sub-clinical annoyance to a severely disabling condition.

Tinnitus may be an accompaniment of sensorineural hearing loss or congenital hearing loss, or it may be observed as a side effect of certain medications.

Tinnitus is prevalent among adults with about 10% of adults in Great Britain report having prolonged, spontaneous tinnitus, and with 1-3% reporting tinnitus severe enough to be disabling. The incidence of tinnitus in the United States is estimated to be 10-15% in adults.

Severe tinnitus is disabling due to the psychological effect of “hearing” sounds or noise continuously. Tinnitus prevents concentration, disrupts or prevents sleep, and may lead to depression.

A wide variety of agents have been used in attempts to treat tinnitus including intravenous administration of local anaesthetics (lidocaine), trans-tympanic injections of local anaesthetics, zinc, steroids, anticonvulsants (carbamazepine), tranquilizers (alprazolam), barbiturates, antidepressants (trimipramine, nortryptyline), and calcium channel blockers (flunarizine). However, these therapies have shown limited efficacy.

It is an object of the present invention to overcome or ameliorate at least one of the disadvantages of the prior art, or to provide a useful alternative.

### SUMMARY OF THE INVENTION

Surprisingly, the present inventors have found that administration of a stromal vascular fraction from adipose tissue treats tinnitus.

In one aspect, the present invention provides a method of treating tinnitus in a subject comprising administering to the subject a stromal vascular fraction from adipose tissue and/or a bone marrow cellular fraction.

In another aspect, the present invention provides use of a stromal vascular fraction from adipose tissue and/or a bone marrow cellular fraction for the manufacture of a medicament for treating tinnitus.

In another aspect, the present invention provides a composition comprising a stromal vascular fraction from adipose tissue and/or a bone marrow cellular fraction for use in treating tinnitus.

In a preferred embodiment, the stromal vascular fraction comprises stem cells.

In a preferred embodiment, the bone marrow cellular fraction comprises stem cells.

In a preferred embodiment, the stem cells are viable stem cell.

In a preferred embodiment, the stem cells are adult stem cells.

In a preferred embodiment, the stromal vascular fraction further comprises extra-cellular matrix.

In a preferred embodiment, the bone marrow cellular fraction further comprises extra-cellular matrix

In a preferred embodiment, the stromal vascular fraction has been cultured and/or expanded.

In a preferred embodiment, the bone marrow cellular fraction has been cultured and/or expanded.

In a preferred embodiment, the stromal vascular fraction is allogeneic.

In a preferred embodiment, the bone marrow cellular fraction is allogeneic.

In a preferred embodiment, the stromal vascular fraction is autologous.

In a preferred embodiment, the bone marrow cellular fraction is autologous.

In a preferred embodiment, the stromal vascular fraction is administered to the subject intravenously, subcutaneously, intra-muscularly or intra-articularly.

In a preferred embodiment, the stromal vascular fraction is added to fat for administration to the subject by fat transfer.

In a preferred embodiment, the bone marrow cellular fraction is administered to a subject intravenously, subcutaneously, intra-muscularly or intra-articularly.

In a preferred embodiment, the bone marrow cellular fraction is added to fat for administration to a subject by fat transfer.

In a preferred embodiment, about 1 million to about 2,000 million stromal vascular fraction (SVF) cells are administered to the subject. For example, about 1 million SVF cells may be administered to the subject, about 5 million SVF cells may be administered to the subject, about 10 million SVF cells may be administered to the subject, about 20 million SVF cells may be administered to the subject, about 30 million SVF cells may be administered to the subject, about 40 million SVF cells may be administered to the subject, about 50 million SVF cells may be administered to the subject, about 100 million SVF cells may be administered to the subject, about 150 million SVF cells may be administered to the subject, about 200 million SVF cells may be administered to the subject, about 250 million SVF cells may be administered to the subject, about 300 million SVF cells may be administered to the subject, about 350 million SVF cells may be administered to the subject, about 400 million SVF cells may be administered to the subject, about 450 million SVF cells may be administered to the subject, about 500 million SVF cells may be administered to the subject, about 600 million SVF cells may be administered to the subject, about 700 million SVF cells may be administered to the subject, about 800 million SVF cells may be administered to the subject, about 900 million SVF cells may be administered to the subject, about 1,000 million SVF cells may be administered to the subject, about 1,100 million SVF cells may be administered to the subject, about 1,200 million SVF cells may be administered to the subject, about 1,300 million SVF cells may be administered to the subject, about 1,400 million SVF cells may be administered to the subject, about 1,500 million SVF cells may be administered to the subject or about 2,000 million SVF cells may be administered to the subject, about 50 million to about 100 million SVF cells may be administered to the subject, about 50 million to about 200 million SVF cells may be administered to the subject, about 50 million to about 300 million SVF cells may be administered to the subject, about 50 million to about 400 million SVF cells may be administered to the subject, about 50 million to about 500 million stromal vascular fraction (SVF) cells may be administered to the subject, about 100 million to about 200 million SVF cells may be administered to the subject, about 100 million to about 300 million SVF cells, about 100 million to about 400 million SVF cells may be administered to the subject, about 100 million to about 500 million SVF cells may be administered to the subject, about 200 million to about 300 million SVF cells may be administered to the subject, about 200 million to about 400 million SVF cells may be administered to the subject, about 200 million to about 500 million SVF cells may be administered to the subject, about 300 million to about 400 million SVF cells may be administered to the subject, about 300 million to about 500 million SVF cells may be administered to the subject, about 400 million to about 500 million SVF cells may be administered to the subject. In a more preferred embodiment, about 10 million to about 1,500 million stromal vascular fraction (SVF) cells are administered to the subject.

In a preferred embodiment, the adipose tissue is lipoaspirate.

In a preferred embodiment, the lipoaspirate is abdominal lipoaspirate.

In a preferred embodiment, the adipose tissue is treated with an enzyme to disaggregate the tissue.

In a preferred embodiment, the enzyme is collagenase or lecithin.

In a preferred embodiment, the adipose tissue is subjected to mechanical agitation and/or centrifugation to disaggregate the tissue.

In a preferred embodiment, the adipose tissue is treated with ultrasound to lyse adipocytes (ultrasonic cavitation) and separate out the adult stem cells from the adipose tissue.

Ultrasonic cavitation may also be used to separate out extracellular matrix from the adipose tissue.

In a preferred embodiment, the probe of an ultrasonic cavitation device (e.g., an ultrasonic processor) is placed into the adipose tissue and the amplitude of the ultrasonic cavitation device is set between about 20% to about 75% and the cycle of the ultrasonic cavitation device is set between about 0.2 to about 0.9, for a period of between about 10 seconds and about 10 minutes. For example, the amplitude of the device is set at about 20%, about 30%, about 40%, about 50%, about 60%, about 70% or about 75%, the cycle of the device is set at about 0.2, about 0.3, about 0.4, about 0.5, about 0.6, about 0.7, about 0.8 or about 0.9, for a period of about 10 seconds, about 20 seconds, about 30 seconds, about 40 seconds, about 50 seconds, about 60 seconds, about 1 minute 10 seconds, about 1 to minute 20 seconds, about 1 minute 30 seconds, about 1 minute 40 seconds, about 1 minute 50 seconds, about 2 minutes, about 2 minutes 10 seconds, about 2 minutes 20 seconds, about 2 minutes 30 seconds, about 2 minutes 40 seconds, about 2 minutes 50 seconds, about 3 minutes, about 3 minutes 10 seconds, about 3 minutes 20 seconds, about 3 minutes 30 seconds, about 3 minutes 40 seconds, about 3 minutes 50 seconds, about 4 minutes, about 5 minutes, about 6 minutes, about 7 minutes, about 8 minutes, about 9 minutes or about 10 minutes.

In a preferred embodiment, the amplitude of the device is set at about 50% and the cycle of the device is set at about 0.4, for a period of about 1.5 minutes to about 2.5 minutes.

In a preferred embodiment, the amplitude, cycle and period are adjusted to prevent the temperature of the adipose tissue (and cells therein) rising to a level at which an effective portion of the cells in the adipose tissue are no longer viable.

In a preferred embodiment, the amplitude, cycle and period are adjusted to prevent the temperature of the adipose tissue (and cells therein) exceeding about 47° C. For example the temperature of the adipose tissue (and cells therein) should be prevented from exceeding about 37° C., exceeding about 38° C., exceeding about 39° C., exceeding about 40° C., exceeding about 41° C., exceeding about 42° C., exceeding about 43° C., exceeding about 44° C., exceeding about 45° C., exceeding about 46° C. or exceeding about 47° C.

In a more preferred embodiment, the amplitude, cycle and period are adjusted to prevent the temperature of the adipose tissue (and cells therein) exceeding about 37° C.

In a preferred embodiment, the stromal vascular fraction is treated with stem cell activators.

In a preferred embodiment, the bone marrow cellular fraction is treated with stem cell activators.

In a preferred embodiment, the stem cell activators are platelet-rich plasma, LED or low-level laser.

In another aspect, the present invention provides a method of treating tinnitus in a subject comprising administering stem cells to the subject.

In another aspect, the present invention provides use of stem cells for the manufacture of a medicament for treating tinnitus.

In another aspect, the present invention provides a composition comprising stem cells for use in treating tinnitus.

In a preferred embodiment, the stem cells are viable stem cells.

In a preferred embodiment, the stem cells are adult stem cells.

In a preferred embodiment, the adult stem cells are obtained from adipose tissue, bone marrow, blood, or a mixture of any two or more thereof.

In a preferred embodiment, the adult stem cells are mesenchymal stem cells.

In a preferred embodiment, the stem cells are embryo-derived stem cells.

In a preferred embodiment, the embryo-derived stem cells are obtained from umbilical cord (Wharton's jelly), umbilical cord blood or placenta, or a mixture of any two or more thereof.

In a preferred embodiment, the stem cells have been cultured and/or expanded.

In a preferred embodiment, the stem cells are autologous.

In a preferred embodiment, the adult stem cells are allogeneic.

In a preferred embodiment, the stem cells are administered to a subject intravenously, subcutaneously, intra-muscularly or intra-articularly.

In a preferred embodiment, the stem cells are added to fat for administration to a subject by fat transfer.

In a preferred embodiment, the stem cells are treated with stem cell activators.

In a preferred embodiment, the stem cell activators are platelet-rich plasma, LED or low-level laser.

As used herein, “adipose” refers to any fat tissue. The adipose tissue may be brown or white adipose tissue. The adipose tissue may be mesenchymal or stromal. Preferably, the adipose tissue is subcutaneous white adipose tissue. The adipose tissue may be from any organism having fat tissue. Preferably the adipose tissue is mammalian, most preferably the adipose tissue is human. A convenient source of human adipose tissue is that derived from surgery, such as liposuction. The source of adipose tissue or the method of isolation of adipose tissue is not critical to the invention.

As used herein, “stromal vascular fraction of adipose tissue” or “SVF” refers to a fraction derived from blood vessels and surrounding tissue found in adipose tissue. As used herein “bone marrow cellular fraction” refers to a cellular fraction derived from bone marrow.

Stem cells found in adipose tissue share many similarities with the stem cells found in bone marrow, including multilineage differentiation capacity (Zuk, P. A. et al. (2002) Human adipose tissue is a source of multipotent stem cells. 13: 4279-4295). As such, it would be anticipated that they have similar therapeutic properties.

The stromal vascular fraction of adipose tissue or the bone marrow cellular fraction may comprise different cell types including, by way of example, mesenchymal stem cells, early mesenchymal/stromal precursor cells, hematopoietic cells, hematopoietic stem cells, platelets, Kupffer cells, osteoclasts, megakaryocytes, granulocytes, NK cells, endothelial precursor or progenitor cells, CD34+ cells, Stro-1+ cells, CD29+ cells, CD166+ cells, Thy-1+ or CD90+ stem cells, CD44+ cells, immune cells such as monocytes, leukocytes, lymphocytes, B and T cells, NK cells, macrophages, neutrophil leukocytes, neutrophils, neutrophil granulocytes, and the like. As used herein, “stromal vascular fraction” includes mesenchymal vascular fractions, mesenchymal fractions, stromal fractions, and the like.

As used herein, “adult stem cell” refers to undifferentiated cells. These cells may be found throughout the body in infants, children and adults. Adult stem cells are capable of long term renewal and differentiation into specialised cell types. An adult stem cell is typically found among differentiated cells in a tissue or organ and can differentiate to yield some or all of the major specialized cell types of the tissue or organ. As used herein, the term “adult stem cell” includes somatic stem cells but excludes embryo-derived cells, such as those obtained from a foetus, an embryo, the placenta, the umbilical cord (Wharton's jelly) or umbilical cord blood.

As used herein, “differentiated cell” refers to a cell that has achieved a state of maturation, such that the cell demonstrates biological specialization and/or adaptation to a specific environment and/or function. Typically, a differentiated cell is characterized by expression of genes that encode differentiation-associated proteins in that cell. For example expression of GALC protein in a leukocyte is a characteristic of a differentiated leukocyte.

As used herein, “mesenchymal stem cell” refers to stromal or mesenchymal cells or early mesenchymal/stromal precursor cells which are multipotent and can serve as stem cell-like precursors to a variety of different cell types such as, but not limited to, adipocytes, osteocytes, chondrocytes, muscle and neuronal/glial cell lineages. Mesenchymal stem cells make up a subset population derivable from, for example, adipose tissue and bone marrow. As used herein, the term “mesenchymal stem cell” includes within its scope stromal stem cells, marrow stromal cells, multipotent stromal cells, mesenchymal precursor cells, mesenchymal progenitor cells, stromal precursor cell, stromal progenitor cells, early mesenchymal precursor cells, early mesenchymal progenitor cells, early stromal precursor cell, early stromal progenitor cells, and the like.

As used herein, “precursor cell”, “progenitor cell” and “stem cell” are used interchangeably (as they are in the art). They refer either to a pluripotent, or lineage-uncommitted, progenitor cell, which is potentially capable of an unlimited number of mitotic divisions to either renew itself or to produce progeny cells which will differentiate into the desired cell type.

As used herein, “multipotent”, “multipotential” or “multipotentiality” refer to the capability of a stem cell to differentiate into more than one type of cell.

As used herein, “allogeneic”, insofar as it refers to allogeneic adipose tissue, allogeneic stromal vascular fraction from adipose tissue, allogeneic bone marrow, allogeneic bone marrow cellular fraction, allogeneic cells or allogeneic stem cells, refers to any adipose tissue, stromal vascular fraction from adipose tissue, bone marrow, bone marrow cellular fraction, cells or stem cells derived from another individual of the same species.

As used herein, “autologous”, insofar as it refers to autologous adipose tissue, autologous stromal vascular fraction from adipose tissue, autologous bone marrow, autologous bone marrow cellular fraction, autologous cells or autologous stem cells, refers to any adipose tissue, stromal vascular fraction from adipose tissue, bone marrow, bone marrow cellular fraction, cells or stem cells derived from an individual and re-introduced to the same individual.

As used herein, the terms “platelet-rich plasma” or “PRP” refer to a blood fraction in which platelets and their associated growth factors are concentrated relative to the source from which the y are derived. In humans, the typical baseline blood platelet count is approximately 200,000 per μL and PRP can have a platelet count that is roughly five-fold higher (Marx, Journal of Oral and Maxillofacial Surgery 2004; 62 (4): 489-96). Several different preparations of PRP may be useful in the invention including, but not limited to, PRP without leukocytes, leukocyte-rich-PRP, platelet rich fibrin and degranulated PRP (Mazzocca et al., J Bone Joint Surg Am 2012; 94:308-316). These different preparations are similar in that a mix of growth factors and cytokines are released from platelets in vitro or in vivo and are produced by well known means (Mazzocca et al., J Bone Joint Surg Am 2012; 94:308-316). Many commercial PRP extraction kits are readily available (Castillo et al., Am K Sports Med 2011; 39(2): 266 to 271).

Heterologous PRP may be obtained from any source, including commercial sources or blood banks.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE FIGURES

FIG. 1: FACS analysis of cells obtained from ultrasonic cavitation-treated adipose tissue-cell count by fluorescent nuclei of 40 grams of adipose tissue.

FIG. 2: Giemsa-stained colonies of cells grown from ultrasonic cavitation-treated adipose tissue.

FIG. 3: Cell culture of mesenchymal stem cells grown from ultrasonic cavitation-treated adipose tissue.

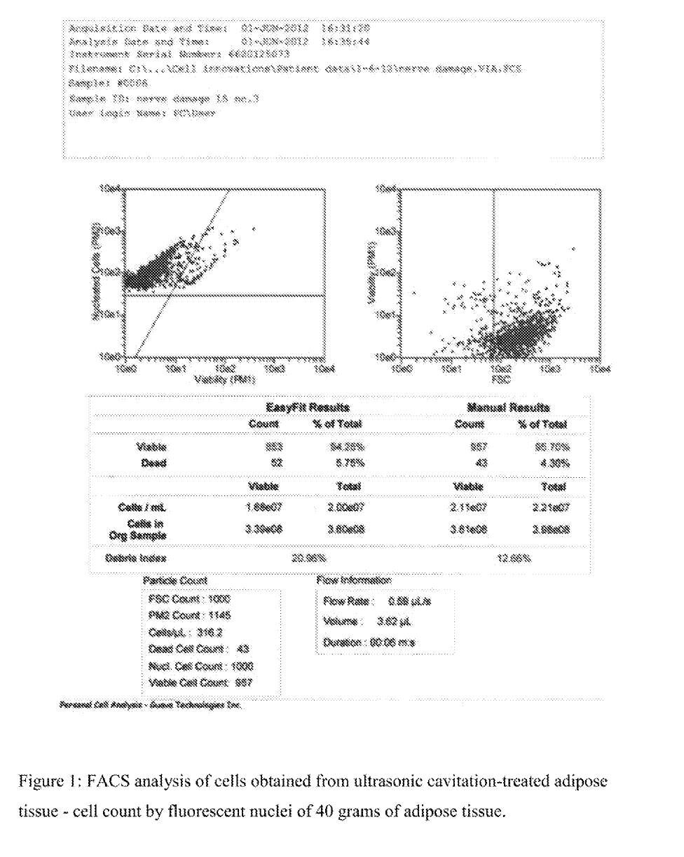

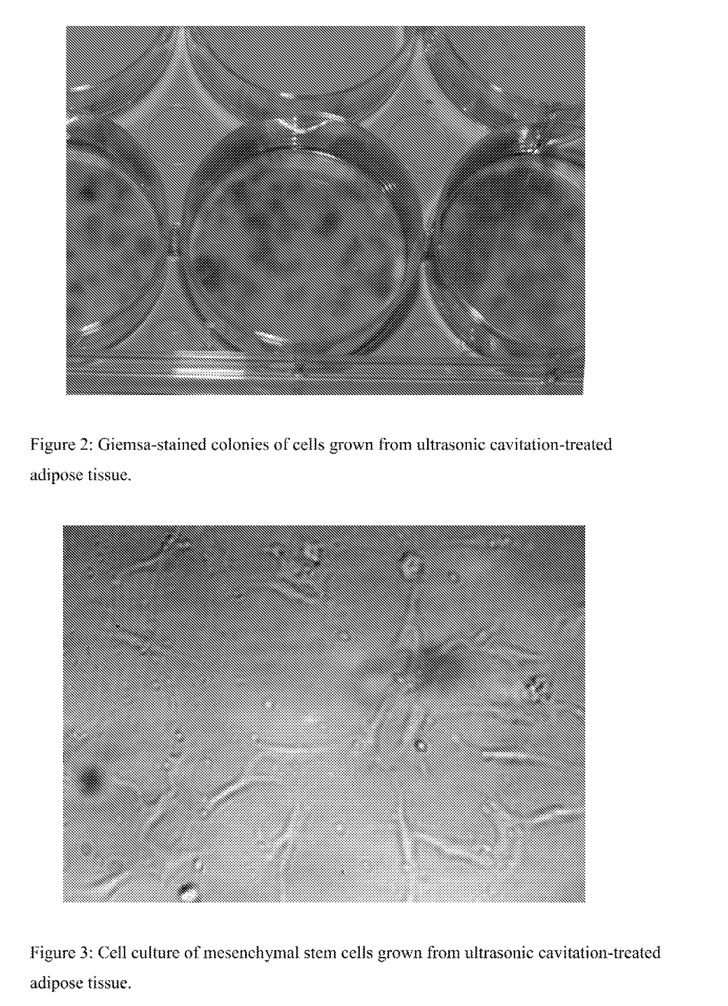

### DETAILED DESCRIPTION OF THE PREFERRED EMBODIMENT

This invention is based on the surprising finding that administration of a stromal vascular fraction from adipose tissue treats tinnitus.

Adipose tissue may be obtained via liposuction surgery, aspiration of fat or isolated by other surgical methods (Biyo Seikei Shujutsu Practice 2 (Cosmetic Operation Practice 2), ed. Masanari ICHIDA, Ryusaburo TANINO, and Yoshiaki HOSAKA, published by BUNKODO, pp. 429-469). Bone marrow may be extracted from the sternum, posterior ilium, or anterior ilium using established techniques.

The adipose tissue or bone marrow may be obtained from the subject who is to be treated. Alternatively, the adipose tissue or bone marrow may be obtained from an allogeneic donor, i.e., an individual that is tissue and/or immune compatible with the subject to be treated. Those of ordinary skill in the art can readily identify allogeneic donors using standard techniques and criteria.

A stromal vascular fraction may be separated from adipose tissue by means of ultrasonic cavitation and/or enzyme treatment and/or mechanical agitation (Locke et al., ANZ J Surg, 2009; 79: 235-244).

Ultrasonic cavitation employs a probe that is placed into contact with the adipose tissue so as to explode or lyse most of the fat cells in the adipose tissue and release the stromal vascular fraction and other cells contained therein. The particular ultrasonic cavitation device used is not critical to the invention. One suitable selection is the Vibra-Cell™ device which is a technologically advanced high intensity ultrasonic processor. This device can safely process a wide range of organic and inorganic materials—from microlitres to litres. Other devices which may be used include HIELSCHLER SONIC 200, and SONIC 200.

Adipose tissue in a biologic solution (e.g., phosphate buffered saline solution or normal saline solution) may be placed into a chilled environment (the tissue/cells should not fall below 2° C.). An ultrasonic cavitation device probe is placed into the adipose tissue and the amplitude is set between 20-75%, typically about 50%, cycle between 0.2-0.9, typically 0.4 for about 10 seconds-10 min. The probe may be adjusted at different positions in the tube during the operation. The procedure may occur at room temperature if the amplitude, cycle and period are adjusted to prevent the temperature of the adipose tissue (and cells therein) rising to a level at which the cells become damaged and no longer viable (e.g., exceed about 47° C.) (Roti, Int. J. Hyperthermia, 2008; 24(1): 3-15). The duration of the ultrasonic cavitation may be dependent on the quantity and amount of adipose. In a typical operation the probe is raised and lowered in the adipose tissue (usually in a 50 ml centrifuge tube) for 1 minute and then left for 30 seconds at the top of the adipose tissue. The process may then be repeated if necessary. The sequence and timing of ultrasonic cavitation may vary and is set at a level that assists in maintaining the viability of the stem cells in the stromal vascular fraction. During the process the adipose tissue temperature is prevented from rising to a level at which the cells become damaged or are no longer viable, i.e., ideally the temperature does not rise above 37° C. or, at most, not above 47° C. After ultrasonication there is a thick solution in the tube which may be centrifuged at 800 g/5 min. Centrifugation results in 3 layers—the top lipid layer, the middle floating layer containing extracellular matrix and stromal vascular cells, and a bottom layer of fluid. The top lipid layer is removed and discarded and the remaining contents of the tube mixed well to separate the extra-cellular matrix and normal saline (0.9% NaCl) added. Further centrifugation at 800 g/5 min results in the cells and extra-cellular matrix falling out and pelleting at the bottom of the centrifugation tube. The pellet contains a stromal vascular fraction comprising viable and functional stem cells (including mesenchymal stem cells), and extracellular matrix. The pellet may be filtered through a 100 um filter to remove any large debris.

In another embodiment, the present invention relates to recovering a stromal vascular fraction from adipose tissue, the method comprising treating the adipose tissue with ultrasonic cavitation for a time, amplitude and cycle that maintains the viability of adult stem cells within the stromal vascular fraction. The adipose tissue may be treated with ultrasonic cavitation for a period of about 10 seconds to about 10 min with an ultrasonic device set at amplitude about 20 to about 75% and cycle about 0.2 to about 0.9. The temperature of the adipose tissue should not rise to a level at which an effective number of the cells become damaged and no longer viable (e.g., the temperature of the adipose tissue (or cells therein) should be preferably kept below about 47° C., even more preferably kept below about 43° C., most preferably kept below about 37° C.).

In another embodiment, the method of the invention uses an enzyme, such as collagenase, and agitation to produce an adipose-derived cell suspension which is centrifuged and washed to separate the stromal vascular fraction (Faustini et al., Nonexpanded Mesenchymal Stem Cells for Regenerative Medicine: Yield in Stromal Vascular Fraction from Adipose Tissues, Tissue engineering: Part C, Volume 16, Number 6, 2010 pp 1515-1521).

The stromal vascular fraction or bone marrow cellular fraction may be directly infused in subjects in need thereof by traditional administration routes, such as intravenous administration (Rodriguez et al., International Archives of Medicine, 2012; 5:5), or it can be further processed to purify (and expand in culture if desired) desired cell types such as mesenchymal stem cells, or STRO-1+ cells prior to administration (Zuk et al., Mol Biol Cell, 2002; 13(12):4279-95; Ra et al., Stem Cells and Development, 2011; 20(8): 1297-1308; Bensidhoum et al., Blood, 2004; 103:3313-3319).

Mesenchymal stem cells may be isolated, purified or enriched from the stromal vascular fraction or bone marrow cellular fraction by fractionation using unique cell surface antigens and fluorescence activated call sorting (FACS) for expansion in vitro (Xiao et al., Mol Cell Biochem, 2013; 377(1-2): 107-19.

The stromal vascular fraction or bone marrow cellular fraction, or mesenchymal stem cells isolated therefrom, may be stored for later implantation/infusion (Berz and Colvin (2012). Cryopreservation of Hematopoietic and Non-Hematopoietic Stem Cells—A Review for the Clinician, in New Advances in Stem Cell Transplantation, Taner Demirer (Ed.), ISBN: 978-953-51-0013-3). Moderate to long-term storage in a cell bank is also within the scope of this invention.

At the end of processing, the stromal vascular fraction or bone marrow cellular fraction, or mesenchymal stem cells isolated therefrom, may be loaded into a delivery device, such as a syringe or IV bag, for administration to the recipient by either subcutaneous, intravenous, intramuscular, or intraperitoneal techniques (Wilson et al., Chapter 5: Medical Administration Techniques, Injections, in Prentice Hall Nurse's Drug Guide 2003, ISBN: 978-0130978721). In other words, cells may be placed into the patient by any means known to persons of ordinary skill in the art, for example, they may be injected into blood vessels for systemic or local delivery, into tissue (e.g., cardiac muscle, or skeletal muscle), into the dermis (subcutaneous), into tissue space (e.g., pericardium or peritoneum), or into tissues (e.g., periurethral emplacement), or other location. Preferred embodiments include placement by needle or catheter, or by direct surgical implantation.

The cells may be delivered in association with additives such as a preformed matrix or adipose-derived or stromal-derived extra-cellular matrix.

The stromal vascular fraction or bone marrow cellular fraction, or mesenchymal stem cells isolated therefrom, may be administered alone or in combination with other cells, tissue, tissue fragments, demineralized bone, growth factors such as insulin or drugs such as members of the thiaglitazone family, biologically active or inert compounds, resorbable plastic scaffolds, adipose-derived or stromal-derived lattice and/or extra cellular matrix or other additive intended to enhance the delivery, efficacy, tolerability, or function of the population (Kuraitis et al., Exploiting extracellular matrix-stem cell interactions: A review of natural materials for therapeutic muscle regeneration, Biomaterials, 2012; 33(2): 428-43; King et al., Current applications of mesenchymal stem cells for tissue replacement in otolaryngology—head and neck surgery, Am J Stem Cell, 2012; 1(3):225-238). In certain embodiments of the invention, the cells are administered to a patient with one or more cellular differentiation agents, such as cytokines and growth factors (Augello et al., The Regulation of Differentiation in Mesenchymal Stem Cells, Human Gene Therapy, 2010; 21:1-13). In other embodiments, the cells are treated with platelet-rich plasma; Gentile et al., A comparative translational study: the combined use of enhanced stromal vascular fraction and platelet-rich plasma improves fat grafting maintenance in breast reconstruction, Stem Cells Transl Med, 2012; 1(4): 341-51.

The present invention will now be described in more detail with reference to specific but non-limiting examples describing specific compositions and methods of use. It is to be understood, however, that the detailed description of specific procedures, compositions and methods is included solely for the purpose of exemplifying the present invention. It should not be understood in any way as a restriction on the broad description of the inventive concept as set out above.

### EXAMPLES

### Example 1

### Preparation of Adipose Tissue by Liposuction

An excess amount of Tumescent solution (containing, in one litre of normal saline, 1 mg adrenalin, 800 mg lignocaine and 10 mLs of a 8.4% sodium bicarbonate solution), which exceeds the amount of liposuction to be aspirated prior to the liposuction operation, was infused into hypodermic fat layer (tumescent method). Thereafter a cannula having, for example, 2-3 mm of inner diameter (made of metal with aspirator) was used for the liposuction operation. Liposuction operations are well known in the art and, for example, can be found in Biyo Seikei Shujutsu Practice 2 (Cosmetic Operation Practice 2), ed. Masanari ICHIDA, Ryusaburo TANINO, and Yoshiaki HOSAKA, published by BUNKODO, pp. 429-469, which is incorporated herein by reference in its entirety.

Aspirated fat was washed with saline. About 50 ml to ten litres of washed aspirate may be generated, and the resultant adipose tissue derived cellular materials used for derivation of stromal vascular fractions.

### Example 2

### Preparation of Adipose Tissue by Surgery

Fat tissue was obtained by surgery from human subjects who had given their informed consent. Separation was conducted with techniques well known in the art. Briefly, human fat tissue was aseptically separated from fat tissue suctioned from human subjects who had given their informed consent. The resultant adipose tissue-derived cellular materials are used for derivation of stromal vascular fractions.

### Example 3

### Preparation of a Stromal Vascular Fraction from Adipose Tissue by Collagenase Treatment

1) Between 50-1000 mL of lipoaspirate was obtained from the patient's abdomen using a 3 mm cannula and Modified Klein's solution. The lipoaspirate was rinsed with normal saline and placed in 500 mL centrifuge pots.
2) Collagenase (Serva) (filter sterilized through a 0.22 μm sterile filter) was added to achieve a final concentration of 0.05%.
3) The sample was incubated at 37° C. for 30-90 minutes in a hot water bath, and gently agitated. During the incubation the sample was gently inverted by hand every 15 minutes.
4) Following incubation, the sample was centrifuged at 500 g×5 min. Three layers were present after the centrifugation. The top yellow/clear layer (lipid layer), the white fibrous middle layer and the red/white bottom layer which has a cell pellet at the bottom of the tube.
5) The cell pellets were removed from the pots by drawing up the pellet with a mixing cannula and syringe.
6) The cell pellets were expelled into a 50 mL centrifuge tube and PBS added to 40 mL. The tube contents were aseptically filtered through a 100 um steriflip (Millipore) using a vacuum pump into the 50 mL tube.
7) The filtrate was centrifuged at 500 g for 5 minutes.
8) The supernatant was removed without disturbing the pellet and all cell pellets combined into the one centrifuge tube. The resultant pellet was resuspended and 40 mL PBS added.
9) The cell suspension was centrifuged at 500 g for 5 minutes and the supernatant removed.
10) 20 mL PBS was added and the cell suspension filtered through a 60 μm steriflip (Millipore).
11) The filtrate was centrifuged at 500 g for 5 minutes and the supernatant removed. A sample was removed for cell counting (a sample of 50 μl of well mixed cells is added to 0.4% of trypan blue, mixed and allowed to stand for 1-2 minutes before placing the sample into a chamber of the haemocytometer. Cell count and viability was determined by counting at least 100 cells in the grid area. Viable cells were determined by exclusion of trypan blue).
12) 5-10 mL platelet-rich plasma or normal saline was added.
13) The sample was drawn up into a syringe and injected into normal saline IV 1 litre bag for infusion into the patient

### Example 4

### Preparation of a Stromal Vascular Fraction Comprising Viable Stem Cells by Ultrasonic Cavitation from 25 ml of Lipoaspirate

1) Adipose tissue was derived from liposuction aspirates and 25 ml of aspirate was placed into 50 ml centrifuge tubes.
2) Excess fluid was removed by centrifugation at 200 g/2 minutes to separate out the excess fluid and adipose tissue. The excess fluid at the base of the tube was removed, typically leaving 20 ml of adipose.
3) The ultrasonic cavitation device probe was placed into the adipose tissue and the amplitude set at 50%, cycle 0.4. The probe was raised and lowered for 1 minute and then for 30 seconds at the top for each tube.
4) After ultrasonication a thick solution was observed in the tube and the tissue centrifuged at 800 g/5 min.
5) After centrifugation there were 3 layers—the top lipid layer, the middle floating layer containing extracellular matrix and stromal vascular cells, and a bottom layer of fluid.
6) The top lipid layer was removed and discarded using a mixing cannula and syringe (removal of the lipid layer permits a separation of cells when isotonic fluid is added) and the remaining contents of the tube were mixed well to further disrupt the extra-cellular matrix.
7) An isotonic solution (typically 0.9% saline) was added to the tube to 45 ml and the mix centrifuged at 800 g/5 mins resulting in the cells and extra-cellular matrix falling out and pelleting at the bottom.
8) A large pellet was observed at the bottom of tube containing extracellular matrix and the stromal vascular fraction comprising viable and functional stem cells. Excess adipose was at the top and saline in the middle.
9) The top layer of excess adipose and saline was removed above the pellet until approximately 15 ml of fluid was left above the pellet. The cell pellet was vortexed and filtered through a 100 um filter to remove any large debris.
10) The cell solution was used as is, or further concentrated by centrifugation and removal of excess fluid, or combining multiple samples.
11) A sample was removed for cell counting and viability to ascertain the quantity of cells to be administered to the patient.

### Example 5

### Preparation of a Stromal Vascular Fraction Comprising Viable Stem Cells by Ultrasonic Cavitation from 45 ml of Lipoaspirate

1) Adipose tissue was derived from liposuction aspirates and 45 ml of aspirate was placed into 50 ml centrifuge tubes.
2) Excess fluid was removed by centrifugation at 200 g/2 minutes to separate out the excess fluid and adipose tissue. The excess fluid at the base of the tube was removed, typically leaving 40 ml of adipose.
3) The ultrasonic cavitation device probe was placed into the adipose tissue and the amplitude set at 50%, cycle 0.4. The probe was raised and lowered for 90 seconds and then for 30 seconds 20 ml mark on the tube and then for 30 seconds at the top of each tube.
4) After ultrasonication a thick solution was observed in the tube and the tissue centrifuged at 800 g/5 min.
5) After centrifugation there were 3 layers—the top lipid layer, the middle floating layer containing extracellular matrix and stromal vascular cells, and a bottom layer of fluid.
6) The top lipid layer was removed and discarded using a mixing cannula and syringe (removal of the lipid layer permits a separation of cells when isotonic fluid is added) and the remaining contents of the tube mixed well to further disrupt the extra-cellular matrix.
7) An isotonic solution (typically 0.9% saline) was added to the tube to 45 ml and the mix centrifuged at 800 g/5 mins resulting in the cells and extra-cellular matrix falling out and pelleting at the bottom.
8) A large pellet was observed at the bottom of tube containing extracellular matrix and the stromal vascular fraction comprising viable and functional stem cells. Excess adipose was at the top and saline in the middle.
9) The top layer of excess adipose and saline was removed above the pellet until approximately 15 ml of fluid was left above the pellet. The cell pellet was vortexed and filtered through a 100 um filter to remove any large debris.
10) The cell solution was used as is, or further concentrated by centrifugation and removal of excess fluid, or combining multiple samples.
11) A sample was removed for cell counting and viability to ascertain the quantity of cells to be administered to the patient.

### Example 6

### Preparation of a Bone Marrow Cellular Fraction

Bone marrow was extracted from the sternum, posterior ilium, or anterior ilium using established techniques. Briefly, the site was prepared with Betadine solution and local anaesthesia was placed under the skin. A longer needle was used to identify the midpoint of the iliac crest and deposit 3-4 mL 2% Xylocaine under the periosteum. A “J” needle was inserted into the anterior/posterior iliac wing. The needle was rotated gently into 1 cm of the marrow cavity. The stylet was removed from the needle and a 5-cc syringe attached. Bone marrow was aspirated by retraction of the plunger of the syringe. After 2-3 mL of marrow was collected, the needle was repositioned if more marrow could be obtained.

Bone marrow harvested by the perfusion or aspiration method were centrifuged and suspended in 15 mL of PBS. The bone marrow was placed on 15 mL of Lymphoprep density solution (1.077 g/mL). After centrifugation for 30 minutes at 2,000 rpm at room temperature, the bone marrow cellular fraction was collected from the defined layer at the interface.

### Example 7

### Preparation of Expanded Mesenchymal Stem Cells

Adult stem cell were obtained from adipose tissue or bone marrow by any suitable method and cultured without differentiation using standard cell culture medium (e.g., alphaMEM typically supplemented with foetal calf serum, human serum or serum free medium). Primary cultures were plated at 1×10⁶/100 mm. The cells were expanded for 1-2 passages (but can be passaged up to 7 times) in 5% CO₂ or hypoxic environment. Such cells may be clonally passaged if required. The isolated autologous or allogeneic cells were cultured to a suitable point and viability and yield assessed by standard methods.

### Example 8

### Preparation of Platelet-Rich Plasma

1) 1-4×9 mL Acid citrate dextrose collection tubes were filled with blood to the black dot (vacuum pressure). The blood was drawn using an 18 G needle or larger to avoid activating the platelets by shearing. The contents of the blood tubes were mixed by inverting the tubes 3-4 times.
2) The blood-filled tubes were centrifuged at 450 g×10 min resulting in three layers—red blood cell layer, buffy coat layer and PRP layer (bottom to top).
3) For PRP without leukocytes, the PRP layer was removed from each tube with care taken not to disturb the buffy coat and platelet red blood cell layers.
4) For leukocyte-rich PRP, the PRP and buffy coat layers were removed with care taken not to remove the red blood cell layer.

### Example 9

### Preparation of Concentrated PRP

1) 1-4×9 mL Acid citrate dextrose collection tubes were filled with blood to the black dot (vacuum pressure). The blood was drawn using an 18 G needle or larger to avoid activating the platelets by shearing. The contents of the blood tubes were mixed by inverting the tubes 3-4 times.
2) The blood-filled tubes were centrifuged at 450 g×10 min resulting in three layers—red blood cell layer, buffy coat layer and PRP layer (bottom to top).
3) For PRP without leukocytes, the PRP layer was removed from each tube with care taken not to disturb the buffy coat and platelet red blood cell layers.
4) For leukocyte-rich PRP, the PRP and buffy coat layers were removed with care taken not to remove the red blood cell layer.
5) The PRP was combined and centrifuged for 2000 g/10 min—a small pellet of platelets at the bottom of the tube formed.
6) The top platelet-poor plasma was removed with a transfer pipette down to 1.5 mL and discarded. The pellet was resuspended in the remaining 1.5 mL using the same transfer pipette.

### Example 10

### Preparation of Platelet-Rich Fibrin and Degranulated PRP

1) PRP or concentrated PRP produced according to examples 1 and 2 was clotted by adding calcium gluconate and mixing well—the tube may placed in a hot water bath (37° C.—without shaking) or left at room temperature for longer period of time.
2) The PRP formed a solid gel, which is platelet-rich fibrin.
3) The gel partially dissolves leaving a fluid which is degranulated PRP.

### Example 11

### Administration of a Stromal Vascular Fraction from Adipose Tissue to Patients with History of Tinnitus

Patient 1

The patient reported tinnitus in both ears for a period of 29 years. The patient was treated with autologous SVF by injecting 1,226,500,000 cells into a 500 ml of bag of normal saline and administering the SVF to the patient intravenously over a period of 20 minutes. The patient received a second treatment 20 months later —frozen autologous SVF was thawed, 200,000,000 viable cells were injected into a 500 ml bag of normal saline and administered to the patient intravenously over a period of 20 minutes.

Tinnitus in the right ear significantly improved following the first SVF administration and completely resolved after the second SVF administration.

Tinnitus in the left ear improved with each SVF administration but did not completely resolve.

Patient 2

The patient reported tinnitus in both ears for a period of 5 years.

The patient was treated with autologous SVF by injecting 125,000,000 cells into a 500 ml of bag of normal saline and administering the SVF to the patient intravenously over a period of 20 minutes.

Tinnitus in the right ear resolved completely by the next day after the treatment.

Tinnitus in the left ear did not improve.

1. A method of treating tinnitus in a subject comprising administering to the subject a stromal vascular fraction from adipose tissue and/or a bone marrow cellular fraction.

2. The method according to claim 1 wherein the stromal vascular fraction or the bone marrow cellular fraction comprises stem cells.

3. The method according to claim 2 wherein the stem cells are adult stem cells.

4. The method according to claim 3 wherein the adult stem cells are mesenchymal stem cells.

5. The method according to claim 1 wherein the stromal vascular fraction or the bone marrow cellular fraction further comprises extracellular matrix.

6. The method according to any one of claims 1 to 5 wherein the stromal vascular fraction or the bone marrow cellular fraction has been cultured and/or expanded.

7. The method according to according to any one of claims 1 to 6 wherein the stromal vascular fraction or the bone marrow cellular fraction is allogeneic.

8. The method according to according to any one of claims 1 to 7 wherein the stromal vascular fraction or the bone marrow cellular fraction is autologous.

9. The method according to according to any one of claims 1 to 8 wherein the stromal vascular fraction or the bone marrow cellular fraction is treated with platelet-rich plasma.

10. The method according to according to any one of claims 1 to 9 wherein the stromal vascular fraction or the bone marrow cellular fraction is administered to the subject intravenously.

11. The method according to according to any one of claims 1 to 10 wherein about 1 million to about 2,000 million stromal vascular fraction cells are administered to the subject.

12. A method of treating tinnitus in a subject comprising administering stem cells to the subject.

13. The method according to claim 12 wherein the stem cells are adult stem cells.

14. The method according to claim 13 wherein the adult stem cells are obtained from adipose tissue, bone marrow or blood, or a mixture of any two or more thereof.

15. The method according to claim 13 or claim 14 wherein the adult stem cells are mesenchymal stem cells.

16. The method according to claim 12 wherein the stem cells are embryo-derived stem cells.

17. The method according to claim 16 wherein the embryo-derived stem cells are obtained from umbilical cord (Wharton's jelly), umbilical cord blood or placenta, or a mixture of any two or more thereof.

18. The method according to any one of claims 12 to 17 wherein the stem cells have been cultured and/or expanded.

19. The method according to any one of claims 12 to 18 wherein the stem cells are allogeneic.

20. The method according to any one of claims 12 to 19 wherein the stem cells are autologous.

21. The method according to any one of claims 12 to 20 wherein the stem cells are treated with platelet-rich plasma.

22. The method according to any one of claims 12 to 21 wherein the stem cells are administered to the subject intravenously.

23. Use of a stromal vascular fraction from adipose tissue and/or a bone marrow cellular fraction for the manufacture of a medicament for treating tinnitus.

24. Use of stem cells for the manufacture of a medicament for treating tinnitus.
